SEQUENCE LISTING (29 sequences)

SEQ ID NO: 1 - DNA, 66 bp, Artificial Sequence
  Description of Artificial Sequence Synthetic oligonucleotide
agcttgccgccatgctgtatacctctcagctgttaggactacttctgttttggatctcggcttcga

SEQ ID NO: 2 - DNA, 67 bp, Artificial Sequence
  Description of Artificial Sequence Synthetic oligonucleotide
gatctcgaagcccgagatccaaaacagaagtagtcctaacagctgagaggtatacagcatggcggca

SEQ ID NO: 3 - DNA, 34 bp, Artificial Sequence
  Description of Artificial Sequence Primer
gttgtcggatccagaaaacagctttgaaatgcaa

SEQ ID NO: 4 - DNA, 44 bp, Artificial Sequence
  Description of Artificial Sequence Primer
gttgtttctagattatcactcgagtttgagtaagccaaaggacg

SEQ ID NO: 5 - DNA, 35 bp, Artificial Sequence
  Description of Artificial Sequence Primer
gttgtcggatccaagaaggttggacaagatagaag

SEQ ID NO: 6 - DNA, 27 bp, Artificial Sequence
  Description of Artificial Sequence Primer
ggatattgatgagatctagtgctacag

SEQ ID NO: 7 - DNA, 42 bp, Artificial Sequence
  Description of Artificial Sequence Primer
gaacacagctcctattggatccggtcttttttctctttgcac

SEQ ID NO: 8 - DNA, 90 bp, Artificial Sequence
  Description of Artificial Sequence Primer
cctgcatggatccgatccgccacctccagaacctccacctcctgaaccgcctccccctcttttttctctttgcactgttcttctctttgc

SEQ ID NO: 9 - DNA, 39 bp, Artificial Sequence
  Description of Artificial Sequence Primer
gttattccatggatccggactaatcttacaatgtgcttg

SEQ ID NO: 10 - DNA, 32 bp, Artificial Sequence
  Description of Artificial Sequence Primer
gtacagctaaatagatctgtagtaattaattg

SEQ ID NO: 11 - DNA, 93 bp, Artificial Sequence
  Description of Artificial Sequence Primer
ggtgcatggatccgaacctccaccgccagatccaccgcctcctgaggcaccgccaccactaatgttacaatgtgcttgttgtcttatatctcc

SEQ ID NO: 12 - DNA, 2252 bp, Artificial Sequence
  Description of Artificial Sequence Synthetic HIV-human fusion construct
aagcttgccgccatgctgtatacctctcagctgttaggactacttctgttttggatctcggcttcgagatctatgctccttgggatattgatgatctgtagtgctacagaaaaattgtgggtcacagtctattatggggtacctgtgtggagagaagcaaccaccactctattttgtgcatcagatgctaaagcctatgatacagaggtacataatgtttgggccacacatgcctgtgtacccacagaccccaacccacaagaagtagtattgggaaatgtgacagaaaattttaacatgtggaaaaataacatggtagatcagatgcatgaggatataatcagtttatgggatgaaagcctaaagccatgtgtaaaattaaccccactctgtgttactttaaattgcactaatttgaatatcactaagaatactactaatcccactagtagcagctggggaatgatggagaaaggagaaataaaaaattgctctttctatatcaccacaagcataagaaataaggtaaagaaagaatatgcactttttaatagacttgatgtagtaccaatagaaaatactaataatactaagtataggttaataagttgtaacacctcagtcattacacaggcctgtccaaaggtatcctttcagccaattcccatacattattgtgtcccggctgggtttgcgatgctaaagtgtaacaataagacattcaatggatcaggaccatgcacaaatgtcagcacagtacaatgtacacatggaattaggccagtggtgtcaactcaactgctgttaaatggcagtctagcagaagaagacatagtaattagatctgaaaatttcacagacaatgctaaaaccataatagtacagctaaatgaatctgtagtaattaattgtacaagacccaacaacaatacaagaagaaggttatctataggaccagggagagcattttatgcaagaagaaacataataggagatataagacaagcacattgtaacattagtagagcaaaatggaataacactttacaacagatagttataaaattaagagaaaaatttaggaataaaacaatagcctttaatcaatcctcaggaggggacccagaaattgtaatgcacagttttaattgtggaggggaattcttctactgtaatacagcacaactgtttaatagtacttggaatgttactggagggacaaatggcactgaaggaaatgacataatcacactccaatgcagaataaaacaaattataaatatgtggcagaaagtaggaaaagcaatgtatgcccctcccatcacaggacaaattagatgttcatcaaatattacagggctgctactaacaagagatggaggtaatagtactgagactgagactgagatcttcagacctggaggaggagatatgagggacaattggagaagtgaattatataaatataaagtagtaagaattgaaccaataggagtagcacccaccagggcaaagagaagaacagtgcaaagagaaaaaagagggggaggcggttcaggaggtggaggttctggaggtggcggatcggatccaagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcgagtgataatctagata

SEQ ID NO: 13 - DNA, 2208 bp, Artificial Sequence
  Description of Artificial Sequence Synthetic HIV-human fusion construct
aagcttgccgccatgctgtatacctctcagctgttaggactacttctgttttggatctcggcttcgagatccatgctccttgggatattgatgatctgtagtgctacagaaaaattgtgggtcacagtctattatggggtacctgtgtggagagaagcaaccaccactctattttgtgcatcagatgctaaagcctatgatacagaggtacataatgtttgggccacacatgcctgtgtacccacagaccccaacccacaagaagtagtattgggaaatgtgacagaaaattttaacatgtggaaaaataacatggtagatcagatgcatgaggatataatcagtttatgggatgaaagcctaaagccatgtgtaaaattaaccccactctgtgttactttaaattgcactaatttgaatatcactaagaatactactaatcccactagtagcagctggggaatgatggagaaaggagaaataaaaaattgctctttctatatcaccacaagcataagaaataaggtaaagaaagaatatgcactttttaatagacttgatgtagtaccaatagaaaatactaataatactaagtataggttaataagttgtaacacctcagtcattacacaggcctgtccaaaggtatcctttcagccaattcccatacattattgtgtcccggctgggtttgcgatgctaaagtgtaacaataagacattcaatggatcaggaccatgcacaaatgtcagcacagtacaatgtacacatggaattaggccagtggtgtcaactcaactgctgttaaatggcagtctagcagaagaagacatagtaattagatctgaaaatttcacagacaatgctaaaaccataatagtacagctaaatgaatctgtagtaattaattgtacaagacccaacaacaatacaagaagaaggttatctataggaccagggagagcattttatgcaagaagaaacataataggagatataagacaagcacattgtaacattagtagagcaaaatggaataacactttacaacagatagttataaaattaagagaaaaatttaggaataaaacaatagcctttaatcaatcctcaggaggggacccagaaattgtaatgcacagttttaattgtggaggggaattcttctactgtaatacagcacaactgtttaatagtacttggaatgttactggagggacaaatggcactgaaggaaatgacataatcacactccaatgcagaataaaacaaattataaatatgtggcagaaagtaggaaaagcaatgtatgcccctcccatcacaggacaaattagatgttcatcaaatattacagggctgctactaacaagagatggaggtaatagtactgagactgagactgagatcttcagacctggaggaggagatatgagggacaattggagaagtgaattatataaatataaagtagtaagaattgaaccaataggagtagcacccaccagggcaaagagaagaacagtgcaaagagaaaaaagaccggatccaagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcgagtgataatctaga

SEQ ID NO: 14 - DNA, 2070 bp, Artificial Sequence
  Description of Artificial Sequence Synthetic HIV-human fusion construct
aagcttgccgccatgctgtatacctctcagctgttaggactacttctgttttggatctcggcttcgagatctatgctccttgggatattgatgatctgtagtgctacagaaaaattgtgggtcacagtctattatggggtacctgtgtggagagaagcaaccaccactctattttgtgcatcagatgctaaagcctatgatacagaggtacataatgtttgggccacacatgcctgtgtacccacagaccccaacccacaagaagtagtattgggaaatgtgacagaaaattttaacatgtggaaaaataacatggtagatcagatgcatgaggatataatcagtttatgggatgaaagcctaaagccatgtgtaaaattaaccccactctgtgttactttaaattgcactaatttgaatatcactaagaatactactaatcccactagtagcagctggggaatgatggagaaaggagaaataaaaaattgctctttctatatcaccacaagcataagaaataaggtaaagaaagaatatgcactttttaatagacttgatgtagtaccaatagaaaatactaataatactaagtataggttaataagttgtaacacctcagtcattacacaggcctgtccaaaggtatcctttcagccaattcccatacattattgtgtcccggctgggtttgcgatgctaaagtgtaacaataagacattcaatggatcaggaccatgcacaaatgtcagcacagtacaatgtacacatggaattaggccagtggtgtcaactcaactgctgttaaatggcagtctagcagaagaagacatagtaattagatctgaaaatttcacagacaatgctaaaaccataatagtacagctaaatgaatctgtagtaattaattgtacaagacccaacaacaatacaagaagaaggttatctataggaccagggagagcattttatgcaagaagaaacataataggagatataagacaagcacattgtaacattagtagagcaaaatggaataacactttacaacagatagttataaaattaagagaaaaatttaggaataaaacaatagcctttaatcaatcctcaggaggggacccagaaattgtaatgcacagttttaattgtggaggggaattcttctactgtaatacagcacaactgtttaatagtacttggaatgttactggagggacaaatggcactgaaggaaatgacataatcacactccaatgcagaataaaacaaattataaatatgtggcagaaagtaggaaaagcaatgtatgcccctcccatcacaggacaaattagatgttcatcaaatattacagggctgctactaacaagagatggaggtaatagtactgagactgagactgagatcttcagacctggaggaggagatatgagggacaattggagaagtgaattatataaatataaagtagtaagaattgaaccaataggagtagcacccaccagggcaaagagaagaacagtgcaaagagaaaaaagagggggaggcggttcaggaggtggaggttctggaggtggcggatcggatccagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcgagtgataatctaga

SEQ ID NO: 15 - DNA, 2028 bp, Artificial Sequence
  Description of Artificial Sequence Synthetic HIV-human fusion construct
aagcttgccgccatgctgtatacctctcagctgttaggactacttctgttttggatctcggcttcgagatccatgctccttgggatattgatgatctgtagtgctacagaaaaattgtgggtcacagtctattatggggtacctgtgtggagagaagcaaccaccactctattttgtgcatcagatgctaaagcctatgatacagaggtacataatgtttgggccacacatgcctgtgtacccacagaccccaacccacaagaagtagtattgggaaatgtgacagaaaattttaacatgtggaaaaataacatggtagatcagatgcatgaggatataatcagtttatgggatgaaagcctaaagccatgtgtaaaattaaccccactctgtgttactttaaattgcactaatttgaatatcactaagaatactactaatcccactagtagcagctggggaatgatggagaaaggagaaataaaaaattgctctttctatatcaccacaagcataagaaataaggtaaagaaagaatatgcactttttaatagacttgatgtagtaccaatagaaaatactaataatactaagtataggttaataagttgtaacacctcagtcattacacaggcctgtccaaaggtatcctttcagccaattcccatacattattgtgtcccggctgggtttgcgatgctaaagtgtaacaataagacattcaatggatcaggaccatgcacaaatgtcagcacagtacaatgtacacatggaattaggccagtggtgtcaactcaactgctgttaaatggcagtctagcagaagaagacatagtaattagatctgaaaatttcacagacaatgctaaaaccataatagtacagctaaatgaatctgtagtaattaattgtacaagacccaacaacaatacaagaagaaggttatctataggaccagggagagcattttatgcaagaagaaacataataggagatataagacaagcacattgtaacattagtagagcaaaatggaataacactttacaacagatagttataaaattaagagaaaaatttaggaataaaacaatagcctttaatcaatcctcaggaggggacccagaaattgtaatgcacagttttaattgtggaggggaattcttctactgtaatacagcacaactgtttaatagtacttggaatgttactggagggacaaatggcactgaaggaaatgacataatcacactccaatgcagaataaaacaaattataaatatgtggcagaaagtaggaaaagcaatgtatgcccctcccatcacaggacaaattagatgttcatcaaatattacagggctgctactaacaagagatggaggtaatagtactgagactgagactgagatcttcagacctggaggaggagatatgagggacaattggagaagtgaattatataaatataaagtagtaagaattgaaccaataggagtagcacccaccagggcaaagagaagaacagtgcaaagagaaaaaagaccggatccagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcgagtgataatctaga

SEQ ID NO: 16 - DNA, 906 bp, Artificial Sequence
  Description of Artificial Sequence Synthetic HIV-human fusion construct
aagcttgccgccatgctgtatacctctcagctgttaggactacttctgttttggatctcggcttcgagatctgtagtaattaattgtacaagacccaacaacaatacaagaagaaggttatctataggaccagggagagcattttatgcaagaagaaacataataggagatataagacaagcacattgtaacattagtggtggcggtggctcaggaggcggtggatctggcggtggaggttcggatccaagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcgagtgataatctaga

SEQ ID NO: 17 - DNA, 864 bp, Artificial Sequence
  Description of Artificial Sequence Synthetic HIV-human fusion construct
aagcttgccgccatgctgtatacctctcagctgttaggactacttctgttttggatctcggcttcgagatctgtagtaattaattgtacaagacccaacaacaatacaagaagaaggttatctataggaccagggagagcattttatgcaagaagaaacataataggagatataagacaagcacattgtaacattagtccggatccaagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcgagtgataatctaga

SEQ ID NO: 18 - DNA, 726 bp, Artificial Sequence
  Description of Artificial Sequence Synthetic HIV-human fusion construct
aagcttgccgccatgctgtatacctctcagctgttaggactacttctgttttggatctcggcttcgagatctgtagtaattaattgtacaagacccaacaacaatacaagaagaaggttatctataggaccagggagagcattttatgcaagaagaaacataataggagatataagacaagcacattgtaacattagtggtggcggtggctcaggaggcggtggatctggcggtggaggttcggatccagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcgagtgataatctaga

SEQ ID NO: 19 - DNA, 684 bp, Artificial Sequence
  Description of Artificial Sequence Synthetic HIV-human fusion construct
aagcttgccgccatgctgtatacctctcagctgttaggactacttctgttttggatctcggcttcgagatctgtagtaattaattgtacaagacccaacaacaatacaagaagaaggttatctataggaccagggagagcattttatgcaagaagaaacataataggagatataagacaagcacattgtaacattagtccggatccagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcgagtgataatctaga

SEQ ID NO: 20 - protein, 742 aa, Artificial Sequence
  Description of Artificial Sequence Synthetic HIV-human fusion construct
MLYTSQLLGLLLFWISASRSMLLGILMICSATEKLWVTVYYGVPVWREATTTLFCASDAKAYDTEVHNVWATHACVPTDPNPQEVVLGNVTENFNMWKNNMVDQMHEDIISLWDESLKPCVKLTPLCVTLNCTNLNITKNTTNPTSSSWGMMEKGEIKNCSFYITTSIRNKVKKEYALFNRLDVVPIENTNNTKYRLISCNTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTRPNNNTRRRLSIGPGRAFYARRNIIGDIRQAHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIEPIGVAPTRAKRRTVQREKRGGGGSGGGGSGGGGSDPRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLE

SEQ ID NO: 21 - protein, 728 aa, Artificial Sequence [sequence omitted]

SEQ ID NO: 22 - protein, 682 aa, Artificial Sequence
  Description of Artificial Sequence Synthetic HIV-human fusion construct
MLYTSQLLGLLLFWISASRSMLLGILMICSATEKLWVTVYYGVPVWREATTTLFCASDAKAYDTEVHNVWATHACVPTDPNPQEVVLGNVTENFNMWKNNMVDQMHEDIISLWDESLKPCVKLTPLCVTLNCTNLNITKNTTNPTSSSWGMMEKGEIKNCSFYITTSIRNKVKKEYALFNRLDVVPIENTNNTKYRLISCNTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTRPNNNTRRRLSIGPGRAFYARRNIIGDIRQAHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIEPIGVAPTRAKRRTVQREKRGGGGSGGGGSGGGGSDPENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLE

SEQ ID NO: 23 - protein, 668 aa, Artificial Sequence
  Description of Artificial Sequence Synthetic HIV-human fusion construct
MLYTSQLLGLLLFWISASRSMLLGILMICSATEKLWVTVYYGVPVWREATTTLFCASDAKAYDTEVHNVWATHACVPTDPNPQEVVLGNVTENFNMWKNNMVDQMHEDIISLWDESLKPCVKLTPLCVTLNCTNLNITKNTTNPTSSSWGMMEKGEIKNCSFYITTSIRNKVKKEYALFNRLDVVPIENTNNTKYRLISCNTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTRPNNNTRRRLSIGPGRAFYARRNIIGDIRQAHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIEPIGVAPTRAKRRTVQREKRPDPENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLE

SEQ ID NO: 24 - protein, 294 aa, Artificial Sequence
  Description of Artificial Sequence Synthetic HIV-human fusion construct
MLYTSQLLGLLLFWISASRSVVINCTRPNNNTRRRLSIGPGRAFYARRNIIGDIRQAHCNISGGGGSGGGGSGGGGSDPRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLE

SEQ ID NO: 25 - protein, 280 aa, Artificial Sequence
  Description of Artificial Sequence Synthetic HIV-human fusion construct
MLYTSQLLGLLLFWISASRSVVINCTRPNNNTRRRLSIGPGRAFYARRNIIGDIRQAHCNISPDPRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLE

SEQ ID NO: 26 - protein, 234 aa, Artificial Sequence
  Description of Artificial Sequence Synthetic HIV-human fusion construct
MLYTSQLLGLLLFWISASRSVVINCTRPNNNTRRRLSIGPGRAFYARRNIIGDIRQAHCNISGGGGSGGGGSGGGGSDPENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLE

SEQ ID NO: 27 - protein, 220 aa, Artificial Sequence
  Description of Artificial Sequence Synthetic HIV-human fusion construct
MLYTSQLLGLLLFWISASRSVVINCTRPNNNTRRRLSIGPGRAFYARRNIIGDIRQAHCNISPDPENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLE

SEQ ID NO: 28 - protein, 6 aa, Human immunodeficiency virus type 1
GPGRAF

SEQ ID NO: 29 - protein, 15 aa, Artificial Sequence
  Description of Artificial Sequence Linker peptide
GGGGSGGGGSGGGGS

(19) United States
(12) Patent Application Publication
(10) Pub. No.: US 2009/0074814 A1
(43) Pub. Date: Mar. 19, 2009

(54) DNA Vaccines Encoding Antigen Linked to a Domain That Binds CD40

(75) Inventors: Jeffrey A. Ledbetter, Shoreline, WA (US); Martha Hayden-Ledbetter, Shoreline, WA (US)

Correspondence Address: Marshall, Gerstein & Borun LLP (Trubion), 233 South Wacker Drive, 6300 Sears Tower, Chicago, IL, 60606-6357, US

(73) Assignee: Jeffrey A. Ledbetter, Shoreline, WA (US)

(21) Appl. No.: 12/233,750

(22) Filed: Sep. 19, 2008

Related U.S. Application Data
(63) Continuation of application No. 11/542,423, filed on Oct. 3, 2006, now abandoned.
Application No. 11/542,423 is a continuation of application No. 09/687,864, filed on Oct. 13, 2000, now Pat. No. 7,118,751.
(60) Provisional application No. 60/159,690, filed on Oct. 14, 1999.

(51) Int. Cl. A61K 39/21; A61K 39/00; C12N 15/00

(52) U.S. Cl. 424/208.1; 424/184.1; 424/277.1; 435/320.1

(57) ABSTRACT

Vaccines that target one or more antigens to a cell surface receptor improve the antigen-specific humoral and cellular immune response. Antigen(s) linked to a domain that binds to a cell surface receptor are internalized, carrying antigen(s) into an intracellular compartment where the antigen(s) are digested into peptides and loaded onto MHC molecules. T cells specific for the peptide antigens are activated, leading to an enhanced immune response. The vaccine may comprise antigen(s) linked to a domain that binds at least one receptor or a DNA plasmid encoding antigen(s) linked to a domain that binds at least one receptor. A preferred embodiment of the invention targets HIV-1 env antigen to the CD40 receptor, resulting in delivery of antigen to CD40 positive cells, and selective activation of the CD40 receptor on cells presenting HIV-1 env antigens to T cells.

### CROSS REFERENCE TO RELATED APPLICATIONS

This application is entitled to the benefit of Provisional Patent Application Ser. No. 60/159,690, filed 1999 Oct. 14.

### BACKGROUND

1. Field of Invention

This invention relates to DNA vaccines, specifically to improved DNA vaccines that induce strong antigen-specific humoral and cellular immune responses.

2. Description of Prior Art

DNA immunization, the inoculation of plasmid DNA encoding a microbial or tumor antigen, is a recent addition to vaccine technology (Donnelly J. J. et al, Ann. Rev. Immunol. 15: 617-648, 1997; Letvin N. L., Science 280: 1875-1879, 1998). Both cellular and humoral immune responses occur after DNA vaccination, and protective immunity against microbial challenge is sometimes induced in experimental animals (Ulmer J. B. et al, Vaccine 12: 1541-1544, 1994; Yokoyama M. et al, J. Virol. 69: 2684-2688, 1995; Xiang Z. Q. et al, Virology 199: 132-140, 1994; Sedegah M. et al, Proc. Natl. Acad. Sci. USA 91: 9866-9870, 1994; Montgomery D. L. et al, DNA Cell Biol. 12: 777-783, 1993). T cell responses, including CD8+ cytotoxic T lymphocyte (CTL) and CD4+ T helper cells, can be stimulated by DNA vaccination in response to antigenic peptides presented by class I and class II MHC molecules (Whitton J. L. et al, Vaccine 17: 1612-1619, 1999). Endogenous protein synthesis allows presentation of foreign antigenic peptides by MHC class I, whereas uptake of soluble protein by APC is required for presentation of peptides by MHC class II. Both arms of the immune response can therefore be induced after DNA vaccination, but the pathways for antigen processing and presentation are distinct for peptides presented by MHC class I or MHC class II. This conclusion is derived from experiments using DNA encoding ubiquitinated protein that is rapidly targeted to intracellular degradation by proteosomes. Ubiquitinated antigen that was degraded so rapidly that intact protein could not leave the cell led to enhanced production of CTL in vivo, but completely eliminated antibody production (Rodriguez F. et al, J. Virol. 71: 8497-8503, 1997; Wu Y. and Kipps T. J., J. Immunol. 159: 6037-6043, 1997). Thus a major limitation of DNA vaccines is their inability to induce strong and sustained humoral immune responses. Strategies for optimization of the cellular immune response to DNA vaccines that do not reduce humoral immune responses are needed.

DNA vaccines for HIV-1 have been tested in animal models and found to induce an immune response that provides protection against challenge only when the virulence of the viral isolate is low. In benign challenge models, chimpanzees were protected from live virus exposure by vaccination with plasmid DNA or by subunit antigens or peptides (Boyer J. D. et al, Nat. Med. 3:526-532, 1997; Kennedy R. C., Nat. Med. 3: 501-502, 1997). However, when highly virulent SIV was tested in rhesus macaques, DNA vaccination was not protective and could only achieve a reduction in virus load even when multiple doses of DNA were inoculated through multiple routes (Lu S. et al, J. Virol. 70: 3978-3991, 1996). Therefore, enhancing the immune response to DNA immunization is an important goal of current AIDS vaccine research. Enhancing the immune response to other DNA vaccines is also desirable in order to provide protection when infected with highly virulent organisms or with a high infectious dose, and to provide long lasting protection. Enhancing the immune response to DNA vaccines encoding tumor antigens is also important for maximizing the anti-tumor response.

One strategy that has been tested is to prime with a DNA vaccine followed by boosting with protein antigen. However, this approach requires construction of multiple vaccines for the same infection or disease, and depends upon multiple injections given in a precise order. It would be desirable to induce protective immunity without needing multiple forms of a vaccine, and without requiring alternating injections of DNA and protein.

Chemical and genetic approaches to enhance the immune response to DNA vaccines have been studied. Chemical adjuvants with some activity include monophosphoryl lipid A (Sasaki S. et al, Infect. Immun. 65: 3520-3528, 1997), saponin QS-21 (Sasaki S et al, J. Virol. 72: 4931-4939, 1998), mannan-coated liposomes (Toda S et al, Immunology 92: 111-117, 1997), and the aminopeptidase inhibitor ubenimex (Sasaki S et al, Clin. Exp. Immunol. 11: 30-36, 1998). Each of these adjuvants modestly enhanced both antibody titers and CTL activity after DNA vaccination in mice. Although the mechanism of action of chemical adjuvants is not fully elucidated, they seem to work by induction of cytokines that amplify responses, by recruitment of macrophages and other lymphoid cells at sites of DNA administration, or by facilitating entry of DNA into host cells (Sasaki S. et al, Anticancer Research 18: 3907-3916, 1998). Several genetic approaches to enhancing responses to DNA vaccines have been tested, including administration of a gene encoding a cytokine (IL2, IL112, GM-CSF, TCA3, MIP-1α) (Chow Y.-H. et al, J. Virol. 71: 169-178, 1997; Hwee Lee A. et al, Vaccine 17: 473-479, 1998; Tsuji T. et al, Immunol. 1.158: 4008-4014, 1997; Rodriguez D. et al, Gen. Virol. 1.80: 217-223, 1999; Tsuji T. et al, Immunology 90: 1-6, 1997; Lu Y. et al, Clin. Exp. Immunol. 115: 335-341, 1999) or a costimulatory adhesion receptor (CD86, CD58, CD54) (Tsuji T. et al, Eur. J. Immunol. 27: 782-787, 1997; Kim J. J. et al, J. Clin. Invest. 103: 869-877, 1999; Iwasaki A. et al, J. Immunol. 158: 4591-4601, 1997). Each of these cytokine and adhesion receptor genes increased immune responses to DNA vaccination, with some treatments enhancing CTL generation only, and some enhancing both CTL and antibody production. However, the levels of enhancement of the immune response to DNA vaccination obtained from these approaches are modest and not sustained, so it is important to find additional ways to enhance the immune response to DNA vaccines.

The CD40 receptor must be activated for an effective cellular or humoral immune response after exposure to antigen (Grewal I. S., and Flavell R. A., Annu. Rev. Immunol 16: 111-135, 1998). This conclusion is derived from multiple findings, including the phenotype of patients with hyper IgM (HIGM) syndrome that results from CD154 genetic defects (Aruffo A. et al, Cell 72: 291-300, 1993; Fuleihan R. et al, Proc. Natl. Acad. Sci. USA 90: 2170-2173, 1993; Korthauer U. et al, Nature 361: 539-541, 1993), the phenotype of mice with CD40 or CD154 gene disruption (Grewal I. S. et al, Science 273: 1864-1867, 1996; Kawabe T. et al, Immunity 1: 167-178, 1994; Renshaw B. et al, J. Exp. Med. 180: 1889-1900, 1994; Xu J. et al, Immunity 1: 423-431, 1994), and the effects of actively blocking CD40 in vivo using inhibitory antibodies to CD154 (Durie F. H. et al, Science 261: 1328-1330, 1993; Foy T. M. et al, J. Exp. Med. 178: 1567-1575, 1993; Foy T. M. et al, J. Exp. Med. 180: 157-163, 1994; Durie F. H. et al, J. Clin. Invest. 94: 1333-1338, 1994; Gerritsse K. et al, Proc. Nat. Acad. Sci. USA 93: 2499-2504, 1996). CD40 is expressed in several cell lineages, including B cells, dendritic cells, monocytes, epithelial cells, and endothelial cells. CD40 transmits signals for each of these cell types that regulates activation and differentiation (Hollenbaugh D. et al, EMBO J. 11: 4313-4321, 1992; Kiener P. A. et al, J. Immunol. 155: 4917-4925, 1995; Celia M. et al, J. Exp. Med. 184: 747-752, 1996; Galy A. H., and Spits H., J. Immunol. 152: 775-782, 1992; Clark E. A., and Ledbetter J. A., Proc. Natl. Acad. Sci. USA 83: 4494-4498, 1986). CD40 is activated by crosslinking during cell to cell contact with cells expressing CD40 ligand (CD154), primarily T cells. While soluble forms of CD154 can stimulate CD40, no attempts have been made to use or modify soluble CD154 to promote immune responses to antigens.

CD40 signals to B cells are required for isotype switching and affinity maturation through somatic mutation (Rousset F. et al, J. Exp. Med. 173: 705-710, 1991). In the absence of CD40 signals, germinal centers, the specialized sites of B cell maturation, are not formed, and B cells are unable to differentiate into IgG producing plasma cells (Foy T. M. et al, J. Exp. Med. 180: 157-163, 1994). Patients with HIGM syndrome are not able to form germinal centers or produce IgG antibodies after antigen challenge, and the same phenotype is seen in knockout mice where CD40 or CD154 is not expressed. The CD40 signal has been shown in vitro to promote survival of surface Ig-activated B cells, and to interact with signals from cytokines to induce immunoglobulin isotype switching to IgG, IgA, and IgE production (Holder M. J. et al, Eur. J. Immunol 23: 2368-2371, 1993; Jabara H. H. et al, J. Exp. Med. 177: 925-935, 1990; Grabstein K. H. et al, J. Immunol. 150: 3141-3147, 1993). In addition, HIGM syndrome patients and CD154 knockout mice have impaired lymphocyte proliferation in response to diphtheria toxoid, tetanus, and Candida, showing that the CD40 signal is required for T cell priming to protein antigens (Grewal I. S., and Flavell R. A., Annu. Rev. Immunol 16: 111-135, 1998; Toes R. E. M. et al, Sem. Immun. 10: 443-448, 1998; Grewal I. S. et al, Nature 378: 617-620, 1995; Ameratunga R. et al, J. Pediatr. 131: 147-150, 1997; Subauste C. S. et al, J. Immunol. 162: 6690-6700, 1999). Expression of CD154 in vivo to enhance immune responses utilized only the cell surface form of the molecule and resulted in significant toxicity in experimental animals, including induction of lethal autoimmune disease and T cell malignancies (Roskrow M. A et al, Leukemia Research 23: 549-557, 1999; Brown M. P. et al, Nature Medicine 4: 1253-1260, 1998).

In neonates, insufficient stimulation of CD40 due to low levels of expression of CD154 by activated T cells has been identified as a factor in the inability of infants to produce IgG antibodies towards bacterial antigens (Nonoyama S. et al, J. Clin. Invest. 95: 66-75, 1995; Fuleihan R. et al, Eur. J. Immunol. 24: 1925-1928, 1994; Brugnoni D. et al, Eur. J. Immunol. 24: 1919-1924, 1994). This suggests that CD40 signals are not ubiquitous and that highly restricted expression of CD154 may limit the extent of CD40 signaling and thus the magnitude and quality of an immune response. Direct evidence in support of this idea comes from a recent study where a modest increase (1.1-2 fold) in expression of cell surface CD154 in the thymus of mice resulted in a >10 fold increase in the antigen-specific antibody response (Prez-Melgosa M. et al, J. Immunol. 163: 1123-1127, 1999). Some evidence suggests that CD40 stimulation may be deficient in HIV-1 infected individuals, since HIV gp120 suppressed the expression of CD154 by activated T cells in vitro, and production of IL12 is defective in HIV-1 positive individuals (Chirmule N. et al, J. Immunol. 155: 917-924, 1995; Taoufik Y. et al, Blood 89: 2842-2848, 1997; Yoo J. et al, J. Immunol. 157: 1313-1320, 1996; Ito M. et al, AIDS Res. Hum. Retroviruses 14: 845-849, 1998; Benyoucef S. et al, J. Med. Virol. 55: 209-214, 1998). In addition, CD40 stimulation of dendritic cells infected with HIV-1 was found to suppress virus replication, suggesting that transmission of HIV-1 from infected dendritic cells during antigen presentation could be blocked by CD40 signals (McDyer J. F. et al, J. Immunol. 162: 3711-3717, 1999). However, a method for stimulation of CD40 on cells actively presenting antigen to T cells while avoiding toxicity from unregulated CD40 stimulation is needed.

CD40 signals to dendritic cells or B cells causes their differentiation from an antigen uptake function to an antigen processing and presentation function (Sallusto D. et al, J. Exp. Med. 182: 389-400, 1995; Cella M. et al, J. Exp. Med. 184: 747-752, 1996; Faassen A. E. et al, Eur. J. Immunol. 25: 3249-3255, 1995). This shift is accompanied by reduction of the MHC class II intracellular compartment, increased expression of MHC class II on the cell surface, secretion of the Th1 regulatory cytokine IL12 and increased expression of CD86 and CD80. After CD40 activation, dendritic cells and B cells are able to more efficiently present antigen and give a critical costimulatory signal through CD28. The production of IL12 leads to enhanced secretion of IFNγ by T cells and suppression of Th2 cytokine production. The CD40 signal is therefore an important mediator of Th1 cellular immunity and CTL induction. However, selective stimulation of CD40 during antigen presentation is needed to enhance immune responses to vaccination.

In addition to B cells and dendritic cells, CD40 is functionally active on other APC's such as monocytes, where CD40 signals prevent cell death from apoptosis and induce expression of adhesion molecules and production of inflammatory cytokines TNFα and IL8 (Kiener P. A. et al, J. Immunol. 155: 4917-4925, 1995). CD40 has also been reported to be expressed and functionally active on thymic epithelial cells (Galy A. H., and Spits H., J. Immunol. 152: 775-782, 1992) and on many kinds of tumor cells, including carcinomas, melanomas, and lymphomas (Ledbetter J. A. et al, In Leucocyte Typing III: White Cell Differentiation Antigens p. 432435, 1987; Oxford University Press, Oxford, U. K.; Paulie S. et al, Cancer Immunol. Immunother. 20: 23-28, 1985). In contrast to most normal cells where the CD40 signal enhances survival, in many malignant cells CD40 actively promotes death by apoptosis. Therefore CD40 is functionally active in all cell types that express the receptor, and CD40 signals are central to fundamental processes of survival and differentiation. Because of the widespread expression of functional CD40, localized stimulation of CD40 positive cells that present specific antigen to T cells is desirable so that only APC involved in the specific immune response are activated.

Studies in CD154 knockout mice have confirmed the importance of CD40 activation for the antigen specific priming of T cells. CD154 deficient mice have an enhanced susceptibility to Leishmania major and Toxoplasma gondii infection, consistent with a central role for CD40 in cellular immunity (Subauste C. S. et al, J. Immunol. 162: 6690-6700, 1999; Campbell K. A. et al, Immunity 4: 283-289, 1996). CTL generation after viral infection in CD154 deficient mice is markedly blunted, and induction of experimental allergic encephalomyelitis (EAE) in response to myelin basic protein does not occur (Grewal I. S. et al, Science 273: 1864-1867, 1996; Grewal I. S. et al, 378: 617-620, 1995). The defect in T cell priming in these models appears to be due to an inability of APC to provide costimulatory signals to T cells (Grewal I. S. et al, Science 273: 1864-1867, 1996; Yang Y. and Wilson J. M., Science 273: 1862-1867, 1996).

Inhibition of CD40 in vivo has been studied in mice using a mAb, MR1, that binds and blocks the CD40 ligand, CD154 (Durie F. H. et al, Science 261: 1328-1330, 1993; Foy T. M. et al, J. Exp. Med. 178: 1567-1575, 1993; Foy T. M. et al, J. Exp. Med. 180: 157-163, 1994; Durie F. H. et al, J. Clin. Invest. 94: 1333-1338, 1994; Gerritsse K. et al, Proc. Nat. Acad. Sci. USA 93: 2499-2504, 1996). These experiments demonstrated that anti-CD154 prevents the induction of autoimmune diseases, including EAE after immunization with myelin basic protein, oophritis after immunization with zona pelucida antigen (ZP3), and spontaneous disease in lupus prone mice (Griggs N. D. et al, J. Exp. Med. 183: 801-807, 1996; Daikh D. I. et al, J. Immunol. 159: 3104-3108, 1997). Anti-CD154 was also effective in preventing both chronic and acute graft versus host (GVH) disease and in preventing rejection of heart allografts after transplantation (Larsen C. P. et al, Nature 381: 434-438, 1996). Thus, CD40 signals are required for T cell responses to antigen, and restriction of the CD40 signal with specific inhibitors is an effective method of limiting T cell priming during an immune response.

The CD40 receptor is therefore a proven target for regulation of antigen specific immunity. While biological inhibitors of CD40 have been studied extensively in mice and in nonhuman primates, there is a need for localized stimulation of CD40 on cells that present antigens to T cells in order to improve the effectiveness of vaccines.

Gp160, the product of the HIV-1 env gene, is cleaved in the Golgi complex into gp120 and gp41 proteins that remain associated through noncovalent interactions. Most neutralizing epitopes of the virus are located on gp120 and gp41, and are expressed by the intact env complex that has been shown to be a trimer (Kwong P. D. et al, Nature 393: 648-659, 1998). Monomeric gp120 can be released from the complex and expose immunodominant epitopes that are non-neutralizing and are located on the internal face of gp120 in the intact trimeric complex (Wyatt R. et al, Nature 393: 705-711, 1998; Broder C. C. et al, PNAS USA 91: 11699-11703, 1994). Thus, stabilization of the env complex is needed for an HIV-1 vaccine in order to preserve conformational epitopes important for neutralization and to mask immunodominant epitopes that are not relevant for neutralization of the env complex.

One attempt to produce a stable, properly folded gp120-gp41 complex was made by altering the cleavage site in gp160 between the gp120 and gp41 domains (Earl P. L. et al, J. Virol. 68: 3015-3026, 1994). By introducing a stop codon before the transmembrane domain of gp41, a soluble molecule composed of gp120 and the extracellular domain of gp41 was produced as a complex that folds properly to bind the CD4 receptor and to express some conformational epitopes. However, this molecule formed dimers and multimers rather than the stable trimers that comprise the native structure of the envelope glycoprotein as revealed in the crystal structure of the gp 120 complex.

Three major sites of gp120 have been identified that are involved in cross-neutralization of diverse viral strains (Wyatt R. et al, Nature 393: 705-711, 1998). The V3 domain was found to express linear and conformational epitopes that can be recognized by antibodies that neutralize HIV-1. Although the V3 domain is a variable region, it contains a central portion shared by many HIV-1 isolates, particularly those found in the United States and Europe. The central portion has been called the principle neutralization epitope and is formed from a linear epitope of the amino acid sequence GPGRAF (Broliden P. A. et al, Proc. Natl. Acad. Sci. USA 89: 461-465, 1992; Broliden P. A. et al, Immunol. 73: 371-376, 1991; Javaherian K. et al, Science 250: 1590-1593, 1990; Javaherian K. et al, Proc. Natl. Acad. Sci. USA 86: 6768-6772, 1989). Conformational epitopes of the V3 loop have also been identified that can be recognized by antibodies that are more broadly neutralizing.

The CD4 binding domain of gp120 is another neutralization site for antibodies directed to HIV-1 env. This domain is a nonlinear, conformational site that depends upon proper folding of gp120 (Kang C.-Y. et al, Proc. Natl. Acad. Sci. USA: 6171-6175, 1991; Lasky L. A. et al, Cell 50: 975-985, 1987). Antibodies can recognize distinct portions of the CD4 binding domain, and may have either type-specific or cross-neutralization properties (Pinter A. et al, AIDS Res. Hum. Retro. 9: 985-996, 1993). Although monomeric gp120 can retain CD4 binding function, a stable trimeric structure of gp120 is thought to be important for masking immunodominant epitopes that are expressed on the internal face of the intact complex (Wyatt R. et al, Nature 393: 705-711, 1998). A third domain of gp120 involved in virus neutralization is exposed upon binding to CD4, and functions to bind the chemokine coreceptor to allow virus entry into the cell (Rizzuto C. D. et al, Science 280: 1949-1953, 1998). Thus a stable trimer of HIV-1 env is needed to present the major cross-neutralization epitopes and to prevent exposure of internal, immunodominant epitopes that do not induce neutralizing antibodies.

CD154 is a TNF-related, type II membrane protein that forms stable trimers (Mazzei G. J. et al, J. Biol. Chem. 270: 7025-7028, 1995). Soluble fusion proteins of human CD154 have been expressed using murine CD8 at the amino terminal side of the CD154 molecule (Hollenbaugh D. et al, EMBO J. 11: 4313-4321, 1992). Single chain Fv (scFv) molecules have also been constructed using heavy and light chain variable regions cloned from the G28-5 hybridoma that produces antibody specific for human CD40 (Ledbetter J. A. et al, Crit. Rev. Immunol. 1.17: 427-435, 1997). Both CD154 and G28-5 scFv fusion proteins retain functional activity as soluble molecules in vitro. However, no use of these molecules to improve the effectiveness of vaccines has been found.

### SUMMARY

For vaccines to be effective, they must induce both humoral and cellular immune responses. This invention describes improved vaccines that target antigens to cell surface receptors. DNA vaccines are a recent addition to immunization technology. However, further optimization of DNA vaccines is needed to induce long-lasting protection against tumor antigens, virulent HIV-1 isolates, and other pathogenic microorganisms. Receptor activation and targeting improves the ability of DNA vaccines to generate strong cellular immunity and high titers of neutralizing antibodies. CD40 is a preferred receptor for targeting and activation. DNA vaccines encoding CD40 ligand (CD154) or a single chain Fv (scFv) specific for CD40, fused with DNA encoding portions of the HIV-1 env protein are preferred embodiments of the invention. A molecule comprising the extracellular domain of HIV-1 env gp160 or env gp120 linked to the extracellular domain of CD154 is a stable trimer that improves immune recognition of HIV-1 env cross-neutralization epitopes. After DNA vaccination, the expression of the fusion protein in vivo results in both activation of the CD40 receptor and direction of HIV-1 env antigens into the endocytic pathway of CD40 positive antigen presenting cells (APC). Internalization of env antigens after binding the CD40 receptor enhances presentation of peptides by MHC molecules. Activation of the CD40 receptor promotes B cell and APC maturation leading to effective antibody production and generation of CD4+ helper T cell and CD8+ CTL activity. The combination of CD40 activation, stabilization of the HIV-1 gp 160 or gp120 env trimer, and enhanced presentation of antigenic peptides by MHC molecules thus improves immune responses to HIV-1 antigens. Protein molecules of the invention can be injected directly into mammals or encoded by DNA vaccines.

### DRAWINGS

FIG. 1.

Schematic representation of fusion proteins that target antigen to cell surface receptors expressed by antigen presenting cells.

A. A fusion protein expressed from a cDNA construct that encodes an antigen domain attached with a linker to a receptor targeting domain. The antigen domain may be attached to the amino terminus of the receptor targeting domain as shown, or may be attached to the carboxy terminus of the receptor targeting domain.

B. A fusion protein expressed from a cDNA construct that encodes the HIV env antigen or a subdomain, is attached to the amino terminus of the CD154 extracellular domain.

C. A fusion protein expressed from a cDNA construct that encodes the HIV env antigen or a subdomain, is attached to the amino terminus of a single chain Fv specific for CD40.

D. A fusion protein expressed from a cDNA construct as in C, except that the scFv that binds CD40 is oriented with the light chain variable region (V_{L}) attached to the carboxy-terminus of the heavy chain variable region (V_{H}).

E. A fusion protein expressed from a cDNA construct that encodes the HIV env antigen or a subdomain, is attached to a camelid variable region (V_{HH}) that binds CD40.

F. A fusion protein expressed from a cDNA construct that encodes the HIV env antigen or a subdomain, is attached to a peptide that binds CD40.

FIG. 2.

A. Sequence of two cDNAs encoding HIV gp120-V3 loop/CD154 long form extracellular domain fusion proteins.

The sequence of a cDNA construct and corresponding fusion protein encoding the HIV V3 loop from gp120 with a (ProAspPro) linker (SEQUENCE ID NO.: 17 [DNA] OR SEQUENCE ID NO.: 25 [FUSION PROTEIN]) or a (Gly₄Ser)₃ linker (SEQ. ID NO.: 16 [DNA] OR SEQ. ID NO.:24 [FUSION PROTEIN]) fused to the CD154 extracellular domain encoded between amino acids 48 (Arg)-261(Leu), with an additional (Glu) residue at the carboxyl end of the protein, not present in wild type CD154. The sequence of the fusion protein is indicated using the three-letter amino acid code convention, above each codon of the open reading frame. Relevant restriction sites are indicated on the drawing and the nucleotides encoding sites at domain fusion junctions are displayed in boldface type, while the first codon of each fused domain is indicated in underlined, italicized type. The protein domains are labeled above the relevant position in the sequence. The nucleotide number is indicated in the left margin with a designation for the PDP linker form or the G4S linker form.

B. Sequence of two cDNAs encoding HIV V3 loop-CD154 short form extracellular domain fusion proteins.

The two HIV V3 loop constructs with alternate linkers, either (ProAspPro) (SEQUENCE ID NO.:19 [DNA] OR SEQUENCE ID NO.: 27 [FUSION PROTEIN]) or (Gly₄Ser)₃ (SEQUENCE ID NO.: 18 [DNA] OR SEQUENCE ID NO.: 26 [FUSION PROTEIN]) were also fused to the short form of the CD154 extracellular domain encoded from amino acids 108 (Glu)-261 (Leu) plus an extra glutamic acid residue at the carboxy terminus, not encoded by wild type CD154. All sequences are labeled as described for FIG. 2A.

FIG. 3.

A. Sequence of two HIV gp120env-CD154 long form extracellular domain cDNA and the predicted fusion proteins.

The sequence of a cDNA construct and corresponding fusion protein encoding the HIV gp120 with a (ProAspPro) linker (SEQ. ID NO.: 13 [DNA] OR SEQ. ID NO.: 21 [FUSION PROTEIN]) or a (Gly₄Ser)₃ linker (SEQ. ID NO.: 12 [DNA] OR SEQ. ID NO.: 20 [FUSION PROTEIN]) fused to the CD154 extracellular domain (Long Form) encoded between amino acids 48 (Arg)-261(Leu)+(Glu). All sequences are labeled as described for FIG. 2A.

B. Sequence of two HIV gp120env-CD154 short form extracellular domain cDNAs and the predicted fusion proteins.

The sequence of a cDNA construct and corresponding fusion protein encoding the HIV gp120 with a (ProAspPro) linker (SEQ. ID NO.: 15 [DNA] or SEQ. ID NO.: 23 [fusion protein]) or a (Gly₄Ser)₃ linker (SEQ. ID NO.: 14 [DNA] or SEQ. ID NO.: 22 [fusion protein]) fused to the short form of the CD154 extracellular domain encoded between amino acids 108 (Glu)-261 (Leu)+(Glu). All sequences are labeled as described for FIG. 2A.

### DESCRIPTION

This invention relates to improved vaccines comprising one or more antigens attached to a domain that targets at least one cell surface receptor. The vaccine may be delivered either as a protein, as a DNA plasmid, or by a viral vector. The expression of the DNA after injection of the plasmid or viral vector in vivo results in the secretion of the antigen(s) attached to a targeting domain, directing the antigen(s) to a cell surface receptor. Receptor-mediated internalization of the antigen into the endocytic compartment of cells that express the receptor enhances the presentation of antigenic peptides by MHC class II molecules that circulate through this compartment.

Presentation of antigenic peptides by MHC class 1 molecules is mediated by the cells expressing the DNA vaccine, and is enhanced in cells that internalize the antigen-targeting domain fusion protein by movement of the fusion protein from the endocytic compartment into the cytoplasm. The activation of antigen-specific CD4+ T cells and CD8+ T cells is increased, resulting in better humoral and cellular immune responses.

The preferred receptor(s) chosen for antigen targeting are those expressed by antigen presenting cells (APC), such as dendritic cells. Desirable receptors for targeting include but are not limited to CD80, CD86, CD83, CD40, CD32, CD64, Flt3, Dec 205, and ICOS ligand. The CD40 receptor is a preferred receptor for antigen targeting, since signals from CD40 regulate activation and differentiation of APC. Fusion proteins of antigen and CD154 (CD40 ligand) combine the functions of antigen targeting and activation of APC by simultaneous delivery of CD40 signals.

The preferred antigen(s) for receptor targeting are HIV-1 and HIV-2 viral antigens, since vaccines have not been effective in protecting against virulent viral isolates. Attachment of HIV-1 gp160 or gp120 extracellular domain to CD154 extracellular domain stabilizes the trimeric structure of HIV-1 env. However, the invention is not limited to HIV env antigens, since improved immune responses to vaccines are needed to provide long-lasting protection against infection with high doses of pathogenic microorganisms or against tumors.

Thus the structure of the invention's main embodiment is a DNA plasmid encoding the extracellular domain of HIV-1 env gp160 attached to the CD154 extracellular domain.

The fusion protein expressed from this DNA plasmid a) stabilizes the trimeric structure of HIV-1 env, b) directs the HIV-1 antigen into the MHC class II compartment of CD40 positive cells, and c) selectively activates the CD40 receptor to increase APC functional activity.

The main embodiment of the invention encodes a stable trimer that expresses the major cross-neutralization epitopes of HIV-1 env while masking the internal env epitopes that are not involved in virus neutralization. Antigenic peptides of HIV env are presented by MHC class I molecules by cells that express the DNA, while antigenic peptides of HIV env are presented by MHC class II molecules in CD40 positive cells that internalize the trimeric antigen-CD154 fusion protein. Activation of the CD40 receptor on cells bound by the antigen-CD154 fusion protein increases the specific immune response due to increased production of IL12 and increased expression of costimulatory molecules CD80 and CD86.

### Operation

An improved DNA vaccine for AIDS comprising the extracellular domain of HIV-1 gp160, HIV-1 gp120, or a subdomain of these antigens fused to the extracellular domain of CD154 is described. Alternative embodiments of the invention use a smaller portion of the CD154 molecule composed of an 18 kDa subunit from Glu-108 to Leu-261 (Mazzei G. J. et al, J. Biol. Chem. 270: 7025-7028, 1995). The extracellular domain of gp160 can also be shortened by removing the gp41 domain, removing the V1 and V2 domains, or mutating the glycosylation sites without damaging the conformational structure of the HIV-1 envelope (Kwong P. D. et al, Nature 393: 648-659, 1998). These changes could further improve the activity of the vaccine, since the V1 and V2 loops, and the carbohydrate structures are thought to be exposed, clade specific epitopes that prevent or dilute the immune response to important cross-neutralization epitopes for diverse clades of HIV-1. Linkers between gp160 and CD154 can also be used. Thus, alternative embodiments of the invention minimize the CD154 domain, remove gp41, V1, V2, or glycosylation sites of gp160. This invention also envisions DNA vaccines comprising other HIV-1 antigens and antigens from alternative isolates of HIV-1, fused to the extracellular domain of CD154.

Delivery of antigen(s) to the CD40 receptor may use anti-CD40 scFv instead of CD154. Single antibody variable regions (V_{HH}) or peptides that bind CD40 are also included in the scope of the invention.

Antigen targeting to receptors is not limited to the CD40 receptor. Alternative receptors preferred for targeting include CD80, CD86, Dec205, ICOS ligand, Flt 3, Fc receptors, and CD83. All cell surface receptors are envisioned by this invention. Receptors may be targeted by ligands, scFv molecules, single variable regions or peptides. Additional methods of attachment of antigen(s) to receptor targeting domains are envisioned, including chemical linkages of subunits, disulfide bonds, or noncovalent attachments such as leucine zipper motifs and the like. The invention contemplates injection of protein, injection of DNA plasmids, or viral vectors encoding the molecules comprising one or more antigens linked to a receptor-binding domain.

Antigens targeted to cell surface receptors are not limited to HIV gp 160 antigens. Other antigens, including tumor antigens, parasite antigens, bacterial antigens, and viral antigens are included in the scope of the invention.

The invention also envisions delivery of antigens to cell surface receptors in order to induce antigen-specific tolerance or nonresponsiveness. For this application, an autoantigen would be chosen and the vaccine would be used to treat autoimmune disease.

The invention also envisions antigen(s) that are natural components of the body, such as tumor-associated antigens, where an immune response to the antigen(s) breaks tolerance to the antigen, resulting in a change in immune homeostasis.

The following examples describe particular embodiments of the invention but are not meant to limit its scope.

### EXAMPLE 1

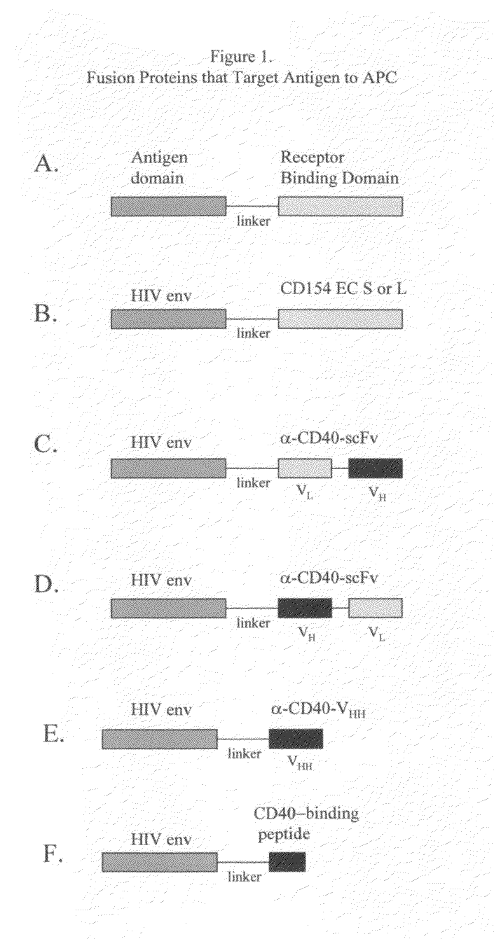

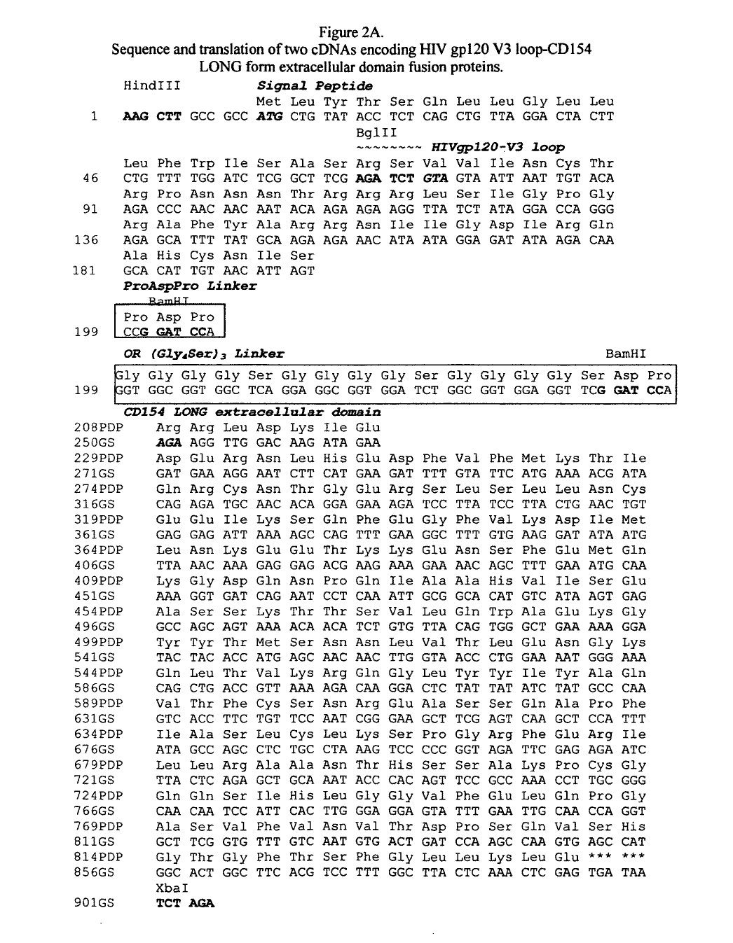

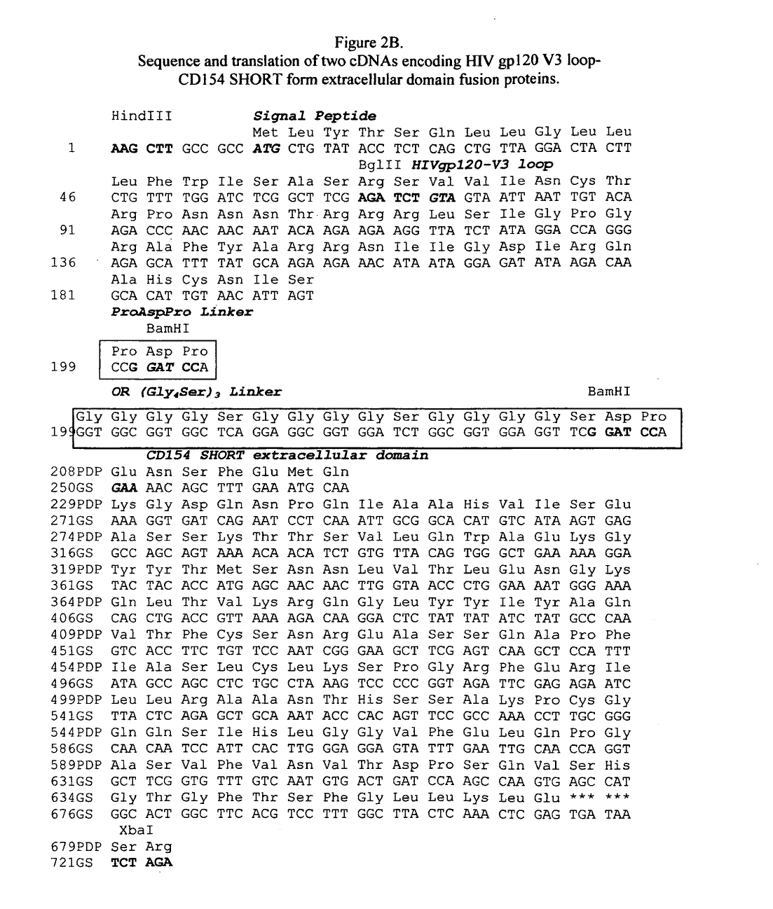

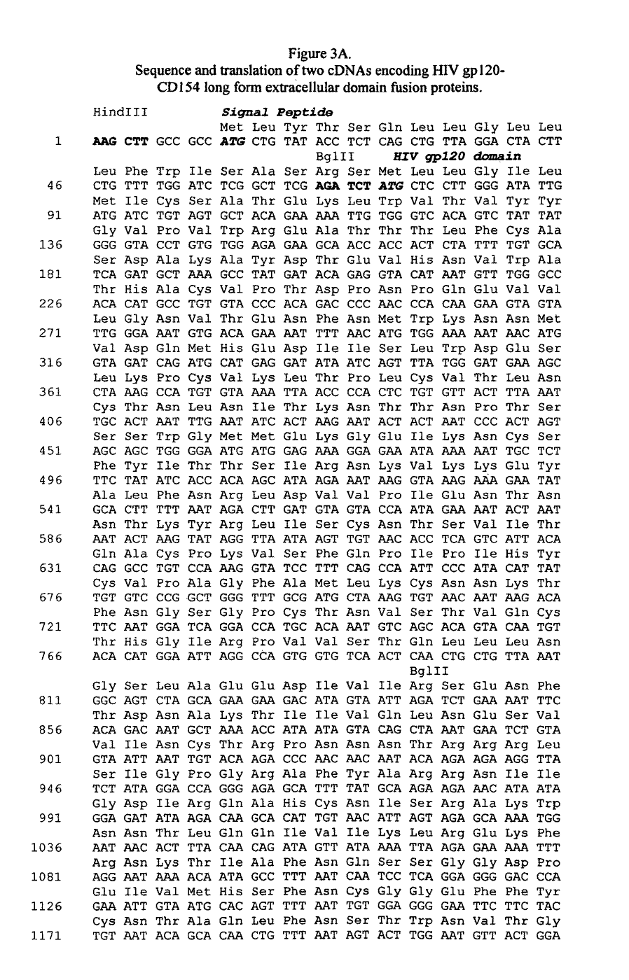

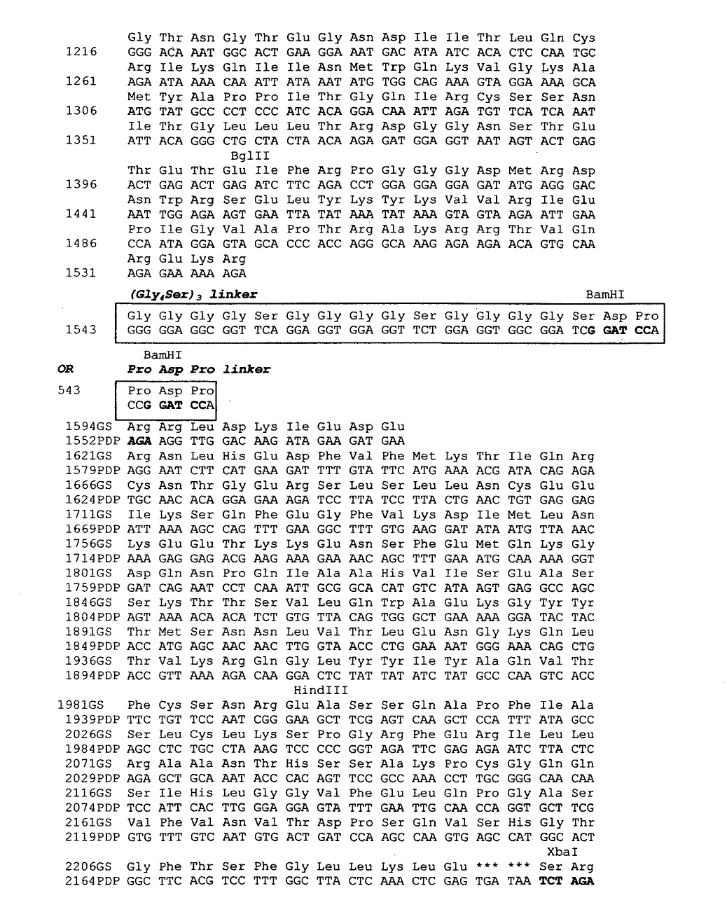

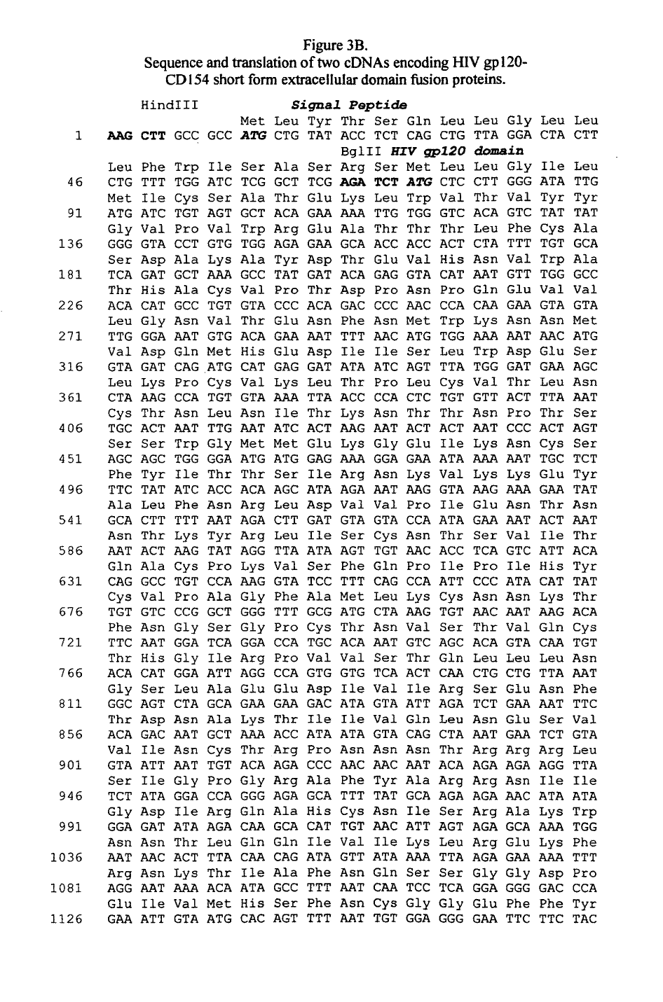

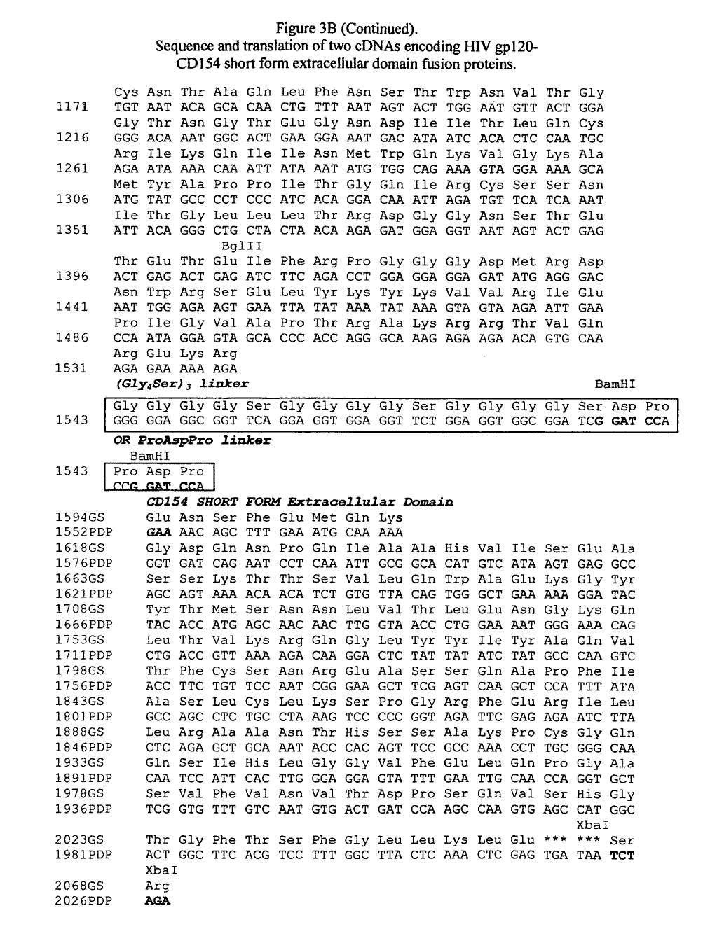

A preferred embodiment of the DNA vaccine includes an amino-terminal secretory signal peptide sequence upstream and adjacent to a cDNA sequence cassette encoding the desired antigen. This molecule is then fused to the extracellular domain of CD154 or to a portion of the extracellular domain of CD154 which retains the ability to bind CD40, or to an scFv targeted to CD40, to create a fusion protein expression cassette that targets the antigen to the antigen presenting cell through the CD40 receptor as diagrammed in FIG. 1. The expression cassette is inserted into an appropriate mammalian expression vector or virus to achieve high level expression of the fusion protein either in vitro or in vivo.

The leader peptide is encoded on complementary oligonucleotides with a single-stranded HindIII cohesive end at the 5′ terminus, and a BglII cohesive end at the 3′ terminus. The sense oligonucleotide is designated SEQUENCE ID NO: 1 or HBLPS and the sequence is as follows: 5′agcttgccgccatgctgtatacctctcagctgttaggactacttctgttttggatctcggcttcga-3′.

The antisense oligonucleotide is designated SEQUENCE ID NO: 2 or HBLPAS and the sequence is as follows: 5′gatctcgaagcccgagatccaaaacagaagtagtcctaacagctgagaggtatacagcatggcggca-3′. The two molecules anneal to one another except at the overhanging nucleotides indicated in boldface type. Alternative embodiments could include other secretory signal peptides or localization sequences.

The extracellular domain of human CD154 was PCR amplified using cDNA generated with random primers and RNA from human T lymphocytes activated with PHA (phytohemagglutinin). Two different fusion junctions were designed which resulted in a short or truncated form (form S4) including amino acids 108 (Glu)-261 (Leu)+(Glu), and a long or complete form (form L2) including amino acids 48 (Arg)-261 (Leu)+(Glu), of the extracellular domain of CD154. The sense primer which fuses the extracellular domain to the targeted antigen includes a BamHI site for cloning that introduces the peptide sequence PDP or (ProAspPro) at the fusion junction and can also encode a linker peptide such as (Gly₄Ser)₃ to separate the antigen from the extracellular domain. The oligonucleotide primers used in amplifying the short form (S4) of the CD154 extracellular domain encoding amino acids 108 (Glu)-261 (Leu)+(Glu) are as follows:

The sense primer is designated SEQUENCE ID NO: 3 or CD154BAM108 and encodes a 34 mer with the following sequence: 5′-gtt gtc gga tcc aga aaa cag ctt tga aat gca a-3′, while the antisense primer is designated SEQUENCE ID NO: 4 or CD154XBA and encodes a 44 mer with the following sequence: 5′-gtt gtt tct aga tta tca ctc gag ttt gag taa gcc aaa gga cg-3′.

The oligonucleotide primers used in amplifying the long form (L2) of the CD 154 extracellular domain encoding amino acids 48 (Arg)-261 (Leu)+(Glu), are as follows: The sense primer is identified as SEQUENCE ID NO: 5 or CD154BAM48 and encodes a 35 mer with the following sequence: 5′-gtt gtc gga tcc aag aag gtt gga caa gat aga ag-3′, while the antisense primer is also SEQUENCE ID NO: 4 or CD154XBA encoding the 44 mer: 5′-gtt gtt tct aga tta tca ctc gag ttt gag taa gcc aaa gga cg-3′.

A variety of different antigens can be encoded on cDNA cassettes to be inserted between the leader peptide cassette and the CD40 targeted domain (such as a truncated or complete CD154 extracellular domain or a CD40 specific scFv). In a preferred embodiment of the invention, the cDNA antigen encoded by the vaccine is the HIV-1 gp 120 or a fragment of this antigen, such as the V3 loop. The primer sets used to amplify the complete gp120 domain include the sense primer SEQUENCE ID NO: 6 or GP120Bgl2f 5′-gga tat tga tga gat cta gtg cta cag-3′ and one of two antisense primers encoding different linkers. Either the antisense primer encoding the ProAspPro linker, identified as SEQUENCE ID NO: 7 or GP120PDPr 5′-gaa cac agc tcc tat tgg atc cgg tct ttt ttc tct ttg cac-3′ or the antisense primer encoding the (Gly₄Ser)₃ linker, identified as SEQUENCE ID NO: 8 or GP120G4Sr 5′-cct gca tgg atc cga tcc gcc acc tcc aga acc tcc acc tcc tga acc gcc tcc ccc tct ttt ttc tct ttg cac tgt tct tct ctt tgc-3′ were used to amplify the gp120 domain with the desired linker attached. PV75 Kgp160(89.6) DNA was used as template in PCR reactions. Alternatively, other isolates or sequence variants of gp 120 or gp160 are available and can be substituted to create novel fusion cassettes. PCR amplification reactions were performed using cloned plasmid DNA as template (approximately 45 ng), 3 mM MgCl₂, 0,3 mM dNTPs, 1/10 volume 10× reaction buffer supplied by the manufacturer, 10 μmol sense primer, 10 μmol antisense primer, and 2.5 units TAQ polymerase (Takara Pharmaceuticals) in a total reaction volume of 50 μl. The amplification profile included an initial 4 minute 94° C. denaturation, followed by a 30 cycle program of 50° C. annealing for 30 seconds, 72° C. extension for 30 seconds, and 94° C. denaturation for 30 seconds. PCR fragments were purified by ethanol precipitation, resuspended in 30 μl ddH₂O and 10 μl was digested with BglII (Roche) restriction endonuclease in a 20 μl reaction volume at 37° C. for 3 hours. Fragments were gel purified, purified using QIAEX kits according to the manufacturer's instructions (QIAGEN, San Diego, Calif.), and ligated along with the annealed leader peptide oligonucleotides to HindIII-BamHI digested expression vector already containing the CD154 extracellular domain as a BamHI-XbaI fragment. Recombinant clones were screened for the correct orientation and presence of inserts, and the resulting positive clones were verified by DNA sequencing using an ABI 310 sequence analyzer and the ABI Prism Dye Terminator Reaction Chemistry. The final fusion cassette encodes the synthetic leader peptide fused to the HIV gp120 domain with either a (ProAspPro) linker or a (Gly₄Ser)₃ linker, and then to the CD154 extracellular domain long (FIG. 3A) or short (FIG. 3B) form to create the embodiments of example 1.

### EXAMPLE 2

In an alternative preferred embodiment, the V1 and V2 domains of gp120 are removed and only the V3 loop domain from HIV gp120 is encoded on a BglII-BamHI fragment and fused to the signal peptide and the CD154 extracellular domain to create the vaccine, as illustrated in FIGS. 2A and B. This antigen domain is separated from the CD154 short (FIG. 2B) or long extracellular domain (FIG. 2A) by a peptide linker encoding the amino acids (ProAspPro), or a longer peptide linker encoding the amino acids (Gly₄Ser)₃.

The V3 loop was PCR amplified from pV75 (gp 89.6), a plasmid containing HIV gp120 from isolate LAV, using the following primer set:

The antisense primer encoding a ProAspPro linker is SEQUENCE ID NO: 9 or V3PDPr 5′-gtt att cca tgg atc cgg act aat ctt aca atg tgc ttg-3′
The sense primer fusing the antigen to the signal peptide is SEQUENCE ID NO: 10 or

### V3Bgl2f

5′-gta cag cta aat aga tct gta gta att aat tg-3′
The antisense primer encoding a (Gly₄Ser)₃ linker is SEQUENCE ID NO: 11 or V3G4Sr 5′-ggt gca tgg atc cga acc tcc acc gcc aga tcc acc gcc tcc tga ggc acc gcc acc act aat gtt aca atg tgc ttg ttg tct tct atc tcc-3′.
Amplification, digestion, purification, and ligation conditions were identical to those described above for the full-length gp120 domain. The final fusion cassettes encode the HIV gp120-V3 loop with either a (ProAspPro) linker or a (Gly₄Ser)₃ linker fused to either the CD154 extracellular domain as diagrammed in FIG. 2A for the long form, and FIG. 2B for the short form of the CD40 binding domain.

Other antigens and linkers can be substituted to create alternative vaccines by construction of the appropriate cDNA cassettes encoding the desired domains and attaching them to the CD154 extracellular domain. Because of the high degree of sequence variation among HIV isolates, alternative sequences might be incorporated as needed to target particular clades. Other viral antigens such as HIV tat or their subdomains can be substituted for the HIV domains described here. Similarly, an alternate APC targeted domain can be substituted for the CD40 binding domain, such as a domain which binds to CD80 or CD86, or to ICOS ligand, or to one of several other cell surface receptors expressed on antigen presenting cells. Surface receptors that internalize readily are preferred over receptors that contain multiple transmembrane domains and do not internalize readily such as G-protein coupled chemokine receptors.

We claim:

1. A vaccine comprising one or more antigens linked to a domain that binds at least one receptor.

2. A vaccine of claim 1 where said receptor is CD40.

3. A vaccine of claim 1 where said domain is CD154 or a portion of CD154.

4. A vaccine of claim 1 where said domain is a single chain Fv that binds CD40.

5. A vaccine of claim 1 where said domain binds to one or more receptors selected from the group consisting of CD80, CD86, CD32, CD64, CD83, ICOS ligand, Flt3, CD10, CD1, CD14, CD15, CD16, CD18, CD19, CD20, CD21, CD22, CD23, CD37, CD38, CD39, CD43, CD56, CD58, CD72, CD75, CD76, CD77, CD78, and cytokine/growth factor receptors.

6. A vaccine of claim 1 where said antigen is HIV-1 gp160 or a portion of HIV-1 gp160.

7. A vaccine of claim 1 where said antigen is a tumor antigen or a microbial antigen.

8. A DNA expression plasmid encoding a vaccine comprising one or more antigens linked to a domain that binds at least one receptor.

9. A DNA expression plasmid of claim 8 encoding a vaccine where said receptor is CD40.

10. A DNA expression plasmid of claim 8 encoding a vaccine where said domain is CD154 or a portion of CD154.

11. A DNA expression plasmid of claim 8 encoding a vaccine where said domain is a single chain Fv that binds CD40.

12. A DNA expression plasmid of claim 8 encoding a vaccine where said domain binds to one or more antigens selected from the group consisting of CD80, CD86, CD32, CD64, CD83, ICOS ligand, Flt3, CD10, CD11, CD14, CD15, CD16, CD18, CD19, CD20, CD21, CD22, CD23, CD37, CD38, CD39, CD43, CD56, CD58, CD72, CD75, CD76, CD77, CD78, and cytokine/growth factor receptors.

13. A DNA expression plasmid of claim 8 encoding a vaccine where said antigen is HIV-1 gp160 or a portion of HIV-1 gp160.

14. A DNA expression plasmid of claim 8 encoding a vaccine where said antigen is a tumor antigen or a microbial antigen.
